(19) United States
(12) Patent Application Publication
(10) Pub. No.: US 2015/0265816 A1
(43) Pub. Date: Sep. 24, 2015

(54) PRECISION BALLOON CATHETER

(71) Applicant: Mark Edward Campbell, Oxford, MS (US)

(72) Inventor: Mark Edward Campbell, Oxford, MS (US)

(21) Appl. No.: 14/663,263

(22) Filed: Mar. 19, 2015

Related U.S. Application Data
(60) Provisional application No. 61/955,247, filed on Mar. 19, 2014.

(51) Int. Cl. A61M 29/02

(52) U.S. Cl. CPC A61M 29/02

(57) ABSTRACT

Intravascular balloon catheters having one or more sets of spatial radiopaque markers circumferentially disposed around an inflatable balloon member or shaft for precise positioning of a stent. The spatial radiopaque markers may be positioned at a place of interest on the inflatable balloon member, such as the edges(s) of the working area or the proximal edge of a stent on the inflatable balloon member. In some embodiments, the spatial radiopaque markers appear as a ring structure when viewed non-orthogonally and as a two-dimensional line structure when viewed orthogonally under fluoroscopy. In some embodiments, a pattern of maximum width and space between a pair of shaft spatial markers is observed when viewed orthogonally under fluoroscopy. These provide the cardiac interventionist great confidence in proper positioning of a stent at an ostial lesion without expensive ultrasound imaging or added techniques and devices. Also disclosed are methods of making and using the same.

### CROSS-REFERENCES TO RELATED APPLICATIONS

This application claims the benefit of U.S. Provisional Application No. 61/955,247 to Mark E. Campbell, filed Mar. 19, 2014, which is hereby incorporated herein by reference in its entirety.

### FIELD OF THE INVENTION

The present invention is generally directed toward an improved balloon catheter device and methods for using same, and more particularly directed toward an improved angioplasty and stent balloon catheter allowing for greater precision in spatial orientation and methods for using same.

### BACKGROUND OF THE INVENTION

Atherosclerosis, which is a lesion characterized by thickening of the blood vessel wall due to lipid, fibrous, and/or calcified deposits that causes a decrease in elasticity, decreased blood flow, and increased blood pressure, is a form of arterial disease that poses a major health problem to patients in the United States and worldwide. It is a chronic disease and often asymptomatic, but if left unchecked, atherosclerosis can present as angina and/or myocardial infarction (heart attack) or stroke. The Centers for Disease Control and Prevention recently estimated the economic cost of heart disease and stroke to be over $312 Billion per year in healthcare costs and lost productivity in the United States alone.

One medical intervention technique for treating atherosclerosis is angioplasty. Angioplasty is the widening of narrowed or obstructed blood vessels, especially arteries due to atherosclerosis. The technique is now commonly performed by deploying a balloon catheter to the site of the lesion and inflating the balloon in a minimally invasive procedure known as percutaneous intervention (“PCI”) with the patient remaining awake. The balloon catheter can also be used to deliver a stent (with or without drug releasing coatings) to help maintain the widened lumen of the vessel. The cardiac interventionist visualizes the PCI procedure by fluoroscopy, along with the use of dyes and/or radiopaque markers on the balloon catheter. Intravenous ultrasound (IVUS) imaging may also be used (or sometimes required) in certain challenging procedures, which adds expense in terms of experienced cardiac interventionists and equipment costs.

Aorto-ostial lesions (lesions at the ostium or “mouth” of a vessel) present a unique challenge in stent placement because the stent must be precisely placed just at the ostium or slightly overhanging into the parent vessel. While aorto-ostial lesions represent less than 10% of coronary artery angioplasty interventions, approximately 95% of all renal lesions are aorto-ostial lesions. Placement of the stent too proximal results in overhang into the larger vessel (usually the aorta), while placement too distal will not widen enough of the lesion. Both can lead to restenosis (recurrence of the narrowing) or the difficulties in the ability to intervene downstream of that branch in the future. Because of the increased challenges and risks, the use of concurrent imaging procedures (IVUS and fluoroscopy) and the development of new add-on devices have added complexity and expense to aorto-ostial angioplasty interventions. There is a need for an improved balloon catheter device that does not require the use of concurrent imaging procedures, added devices, or added complexity to angioplasty of aorto-ostial lesions.

### SUMMARY OF THE INVENTION

The disclosed invention overcomes the shortcomings of the prior known devices and techniques. In one aspect, the present invention provides an inflatable balloon member for angioplasty having a spatial radiopaque marker set circumferentially disposed around said inflatable balloon member.

In another aspect, the present invention provides an intravascular balloon catheter comprising a hollow shaft member for extending a guidewire there through, said shaft member having at least one inner lumen for passing a fluid there through, an inflatable balloon member attached to said shaft member, an inner lumen of said inflatable balloon member in fluid communication with said at least one inner lumen of said shaft member for passing a fluid there through, and a spatial radiopaque marker set circumferentially disposed around said inflatable balloon member.

In a further aspect, the present invention provides a method for making an intravascular balloon catheter with spatial radiopaque markers comprising: forming a hollow shaft member for extending a guidewire there through, said shaft member having at least one inner lumen for passing a fluid there through; forming an inflatable balloon member securely attached to said shaft member and leaving an inner lumen of said inflatable balloon member to be in fluid communication with said at least one inner lumen of said shaft member for passing a fluid there through; and incorporating a spatial radiopaque marker set circumferentially around said inflatable balloon member.

In still another aspect, the present invention provides a method for making an intravascular balloon catheter with spatial radiopaque markers comprising: providing an intravascular balloon catheter with an inflatable balloon member; and incorporating a spatial radiopaque marker set circumferentially around said inflatable balloon member by flexible adhesive.

### BRIEF DESCRIPTION OF THE DRAWINGS

Further advantages of the invention will become apparent by reference to the detailed description of preferred embodiments when considered in conjunction with the drawings:

FIG. 1 depicts an elevated view of a prior art intravascular balloon catheter.

FIG. 2 depicts an elevated view of one embodiment of an intravascular precision balloon catheter of the present invention having proximal radiopaque balloon spatial markers.

FIG. 3 depicts an elevated view of the embodiment depicted in FIG. 2 having proximal radiopaque balloon spatial markers and a stent for delivery to a lesion.

FIG. 4 depicts an elevated view of another embodiment of an intravascular precision balloon catheter of the present invention having proximal and distal radiopaque balloon spatial markers.

FIG. 5 depicts an elevated view of the embodiment depicted in FIG. 4 having proximal and distal radiopaque balloon spatial markers and a stent for delivery to a lesion.

FIG. 6A shows a perspective view (non-orthogonal) of another embodiment of the intravascular precision balloon catheter of the present invention shown under fluoroscopy presenting three possible embedded radiopaque markers from proximal to distal (adhesively fixed set of radiopaque markers, adhesively fixed set of broken ring radiopaque markers, and adhesively fixed continuous ring radiopaque marker); note how the radiopaque markers are seen as a “ring” out of plane when viewed at a non-orthogonal angle.

FIG. 6B shows an elevated view (orthogonal) of the embodiment depicted in FIG. 6A shown under fluoroscopy presenting three possible incorporated radiopaque markers from proximal to distal (adhesively fixed set of radiopaque markers, adhesively fixed set of broken ring radiopaque markers, and adhesively fixed continuous ring radiopaque marker); note how the radiopaque markers “line up” in a plane when viewed at an orthogonal angle.

FIG. 7 depicts an elevated view of yet another embodiment of an intravascular precision balloon catheter of the present invention having a pair of dual proximal and distal shaft radiopaque spatial markers.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

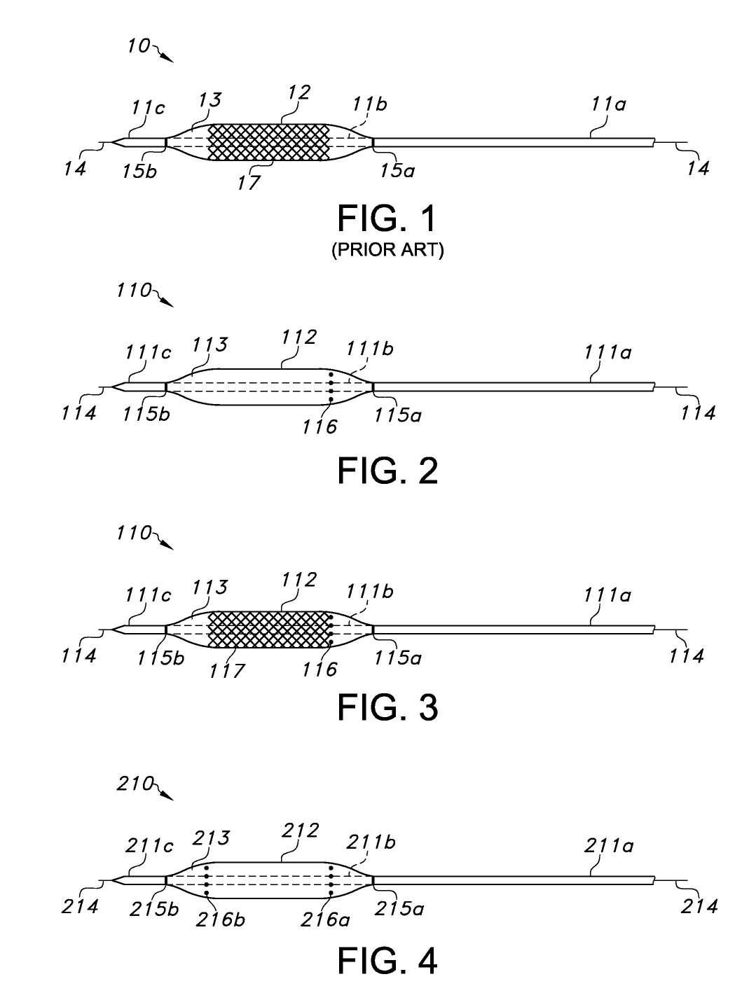

Referring to the drawings, FIG. 1 illustrates an example of a prior art intravascular balloon catheter 10 that can benefit from the improvements disclosed herein. The balloon catheter 10 comprises an elongated shaft 11. Shaft 11 has a proximal portion 11a (defined as the portion of shaft 11 that is proximal relative to the operator), a distal portion 11c (defined as the portion of shaft 11 that is distal relative to the operator), and a balloon portion 11b (defined as the portion of shaft 11 within or under the inflatable balloon member 12). Distal portion 11c is preferably tapered for entry into the patient's arteries. Shaft 11 is disposed on a guidewire 14 by slidable engagement thereon, and may be comprised of one or more tubular members with lumens in liquid communication with balloon 12. The inflatable balloon member 12 is securely attached onto shaft 11. Guidewire 14 can be any catheter guidewire suitable for use in the circulatory system of a patient for delivering a balloon catheter to a lesion site. The shaft 11 and guidewire 14 may be made from or coated with a hydrophilic material to facilitate movement within the patient's vasculature system.

Inflatable balloon member 12 is designed to expand at a lesion site to compress the plaque and thereby widening the patient's artery. To accomplish this, inflatable balloon member 12 is typically made of a non-compliant or semi-compliant polymeric material as known in the art, and/or as described in U.S. Pat. No. 5,304,197 to Pinchuk et al. (hereby incorporated by reference). The inner chamber or lumen 13 of inflatable balloon member 12 is in fluid communication with an interior lumen of shaft 11 such that a fluid can be pumped into and inflate the inner lumen 13. The inflatable balloon member 12 at each end is sealingly connected to shaft 11 at the junctions of shaft portions 11a-11b and at 11b-11c so that the inflating fluid is maintained only within lumen 13. To ensure a proper angioplasty result, the inflating fluid is provided at several times normal blood pressure and only for a very brief period of time. Inflation causes inflatable balloon member 12 to reach a fixed or predetermined size at a given pressure that is proper for the artery and lesion being widened in the angioplasty procedure. Under-inflation by applying less than full pressure will cause inflatable balloon member 12 to not fully reach its predetermined size, which may be beneficial to a given application. The inflatable balloon member 12 and its seals must be capable of withstanding such extreme pressures, hold the predetermined size, and not burst.

In a typical angioplasty procedure, an experienced cardiac interventionist performs a percutaneous intervention (“PCI”), which is a minimally invasive procedure performed in a cath lab while the patient remains conscious but mildly sedated to relax the patient. The cardiac interventionist places an introducer needle into the femoral artery in the leg, although arteries in the arm or neck can also be used depending on the location of the lesion and other factors not important for a 7 discussion of the present inventions. Next, a sheath introducer is inserted. The catheter is then placed through the sheath introducer. The cardiac interventionist will then usually study the lesion(s) of the patient by injecting radiopaque dyes that allow contrast of the arteries on X-ray and/or fluoroscopy images to determine the extent of lesion(s), the type and positioning of the lesion(s), and the type and size of balloon catheter 10 needed. An intravenous ultrasound (IVUS) imaging catheter may also be used to study the lesion(s) of the patient in situ.

The guidewire 14 is then inserted into the catheter by sliding through shaft 11 into a coronary artery to be treated. The cardiac interventionist will visualize the path and positioning of the shaft 11 and guidewire 14 by observing radiopaque markers 15 located on each by fluoroscopy, an X-ray imaging technique using video rather than still images. For example, guidewire 14 usually has a radiopaque marker at its tip only (not shown for simplicity), while shaft 11 usually has a proximal shaft radiopaque marker 15a and a distal shaft radiopaque marker 15b. Proximal shaft radiopaque marker 15a and distal shaft radiopaque marker 15b are located on shaft 11 at the proximal and distal junctions, respectively, of the inflatable balloon member 12 to indicate the extreme ends of balloon 12 along shaft 11. The markers 15a & 15b are usually formed on shaft 11 by adhesive, mechanical means (such as clamping or fixing the marker material around the shaft 11 itself), or embedded into the shaft 11 material during manufacturing of the balloon 12 and/or shaft 11 Radiopaque markers 15a & 15b can be made of any radiopaque material, including dense metals (such as gold, platinum, tantalum, or alloys thereof) or polymeric materials containing radiopaque fillers (such as barium sulfate, bismuth compounds, and tungsten).

While viewing the marker(s) via fluoroscopy, the cardiac interventionist will guide the guidewire 14 to the lesion site, and it is extended through the lesion site. Then, shaft 11 with inflatable balloon member 12 is inserted along guidewire 14 and guided to the lesion site. In most cases, the inflatable balloon member 12 is positioned through the lesion site and then inflated as described above, expanding the lesion site (plaque and artery wall). The cardiac interventionist may also deliver optional stent 17 via an inflatable balloon member 12 with the stent positioned on the outside of the inflatable balloon member 12. A stent 17 is an expandable wire mesh tube that is delivered via a balloon catheter 10. Stent 17 may be bare wire or coated with a drug releasing coating designed to prevent or limit restenosis (a re-narrowing of the lesion site). When the cardiac interventionist inflates balloon 12 briefly, the stent 17 will expand against the widened lesion site and/or artery wall and remain a permanent implant in the patient at the site. The cardiac interventionist will use the markers 15 as described above to position the stent 17 prior to inflation and stent expansion.

Some angioplasty procedures, such as ostial lesions, are very challenging because they require very precise positioning of the inflatable balloon member 12 and stent 17. An ostial lesion is a lesion located at the ostium or mouth of a branch artery stemming from a parent vessel, such as the aorta (aorta-ostial lesions). Proper placement of a stent 17 at an ostium is generally understood to be about 0.5 mm overhang in the parent vessel if the target vessel is positioned at a 90° angle from the parent vessel. For target vessels having less than a 90° angle from the parent vessel, proper placement is generally considered to be at or within 0.5 mm of the ostium at the closest point and overhang into the parent vessel with the remainder of the proximal edge of the stent 17 as necessary. Improper placement of a stent 17 at an aorto-ostial lesion may result in excessive stent 17 overhang into the aorta, which can cause plaque shift into the aorta (or parent vessel) and/or make it difficult to conduct a subsequent intervention downstream of the affected artery. On the other hand, positioning the stent 17 too far from the ostium will not sufficiently treat the blockage, which can allow for restenosis and/or make it difficult to conduct a subsequent intervention downstream of the affected artery. At branch arteries having a <75° angle from the aorta (parent vessel), the problem is compounded in that proper placement requires some overhang in the aorta (parent vessel). See, e.g., P. Jokhi & N. Curzen. Percutaneous coronary intervention of ostial lesions. Eurolntervention 2009; 5:511-514, hereby incorporated by reference. Thus, precise positioning of inflatable balloon member 12 and stent 17 is critical in ostial lesion procedures.

A proximal shaft radiopaque marker 15a (the marker that will be closest to the ostium) is insufficient for precise placement aorto-ostial lesion procedures because the resulting two-dimensional fluoroscopy imaging does not accurately reflect the plane of the ostium of the target branch artery unless viewed orthogonally (i.e., the plane of the ostium is parallel with the viewer's line of sight). Furthermore, because the fluoroscopy image is two-dimensional, the cardiac interventionist may incorrectly believe the image is orthogonal when it is not, and a difference of just a few degrees can result in an improper position of the stent 17. Therefore, aorto-ostial lesions are among the most challenging angioplasty procedures to perform.

Due to these challenges and the requirement for precise positioning of a stent 17 in aorto-ostial lesions, some techniques and a couple of ostium-specific devices have been developed to help ensure proper positioning of a stent 17 at the ostium. For example, IVUS imaging is often performed using a second guided catheter to help an experienced cardiac interventionist in precise placement of the stent 17. IVUS imaging requires the use of expensive equipment, a disposable ultrasound catheter, and additional training, which leads to added costs for the patient. Some cardiac interventionists have developed techniques, such as “stent draw-back” and “Szabo” techniques to place stents 17 at an ostium, but these require special training and additional catheter equipment, while also presenting several inherent disadvantages such as, but not limited to, damage to the stent delivery balloon catheter or placement of the stent 17 more than 1 mm from the ostium. The BULLSEYE™ renal ostial stent system was developed to incorporate a proximal balloon and special stent with wider proximal end to be expanded at a renal aorto-ostial lesion site. This device can still result in excessive overhang of the special stent in the aorta. Also, the OSTIAL PRO™ stent system was developed to provide an additional device to prevent the guide catheter from entering the target vessel and mark the plane of the ostium. Because aorto-ostial planes are rarely perfectly flat, the device may be prone to error by giving a false impression of an orthogonal view of the ostial lesion site. Thus, all of these techniques and devices add additional complexity and expense to the angioplasty procedures at an aorto-ostial lesion, while not overcoming all of the issues that make ostial lesions challenging to treat.

Now referring to FIGS. 2 & 3, an exemplary embodiment of the intravascular precision balloon catheter 110 of the present invention is shown, with (FIG. 3) and without (FIG. 2) stent 117. The components of precision balloon catheter 110 are essentially the same as that described above. For example, hollow shaft 111 is slidably engaged over a guidewire 114 to deliver an inflatable balloon member 112 with inner lumen 113 that is in fluid communication with at least one inner lumen of shaft 111. Shaft 111 has a proximal portion 111a (defined as the portion of shaft 111 that is proximal relative to the operator), a distal portion 111c (defined as the portion of shaft 111 that is distal relative to the operator), and a balloon portion 111b (defined as the portion of shaft 111 within or under the inflatable balloon member 112). Distal portion 111c is preferably tapered for entry into the patient's vessels. Shaft 111 may also comprise optional proximal shaft radiopaque marker 115a and a distal shaft radiopaque marker 115b.

Inflatable balloon member 112 is designed to expand at a lesion site to compress the plaque and thereby widening the patient's vessel. Inflatable balloon member 112 can be made of a non-compliant or semi-compliant polymeric material, as described above. The inflatable balloon member 112 at each end is sealingly connected to shaft 111 at the junctions of shaft portions 111a-111b and at 111b-111c so that the inflating fluid is maintained only within lumen 113. To ensure a proper angioplasty result, the inflating fluid is provided at several times normal blood pressure and only for a very brief period of time. Inflation causes inflatable balloon member 112 to reach a fixed or predetermined size at a given pressure that is proper for the artery and lesion being widened in the angioplasty procedure. Under-inflation by applying less than full pressure will cause inflatable balloon member 112 to not fully reach its predetermined size, which may be beneficial to a given application. The inflatable balloon member 112 and its seals must be capable of withstanding such extreme pressures, hold the predetermined size, and not burst.

As can be appreciated in FIGS. 2 & 3, a key difference between the precision balloon catheter 110 and balloon catheter 10 is the “ring” set of balloon spatial radiopaque markers 116 placed circumferentially around balloon 112. When viewed orthogonally (as shown in FIGS. 2 & 3), balloon spatial radiopaque markers 116 define a plane that is perpendicular to the length of shaft 111b associated with balloon 112. Balloon spatial radiopaque markers 116 may be positioned anywhere on balloon 112, but preferably balloon spatial radiopaque markers 116 are positioned at a site of interest to the cardiac interventionist. For example, such a site of interest might be the proximal edge of the working area of balloon 112 (defined as the portion of the balloon that, when inflated, is designed to directly participate in the widening of the target vessel; see FIG. 2) or the proximal end of stent 117 as placed on balloon 112 (see FIG. 3). Depending on the particular application site, stent 117 may or may not extend to the distal edge of the working area of balloon 112.

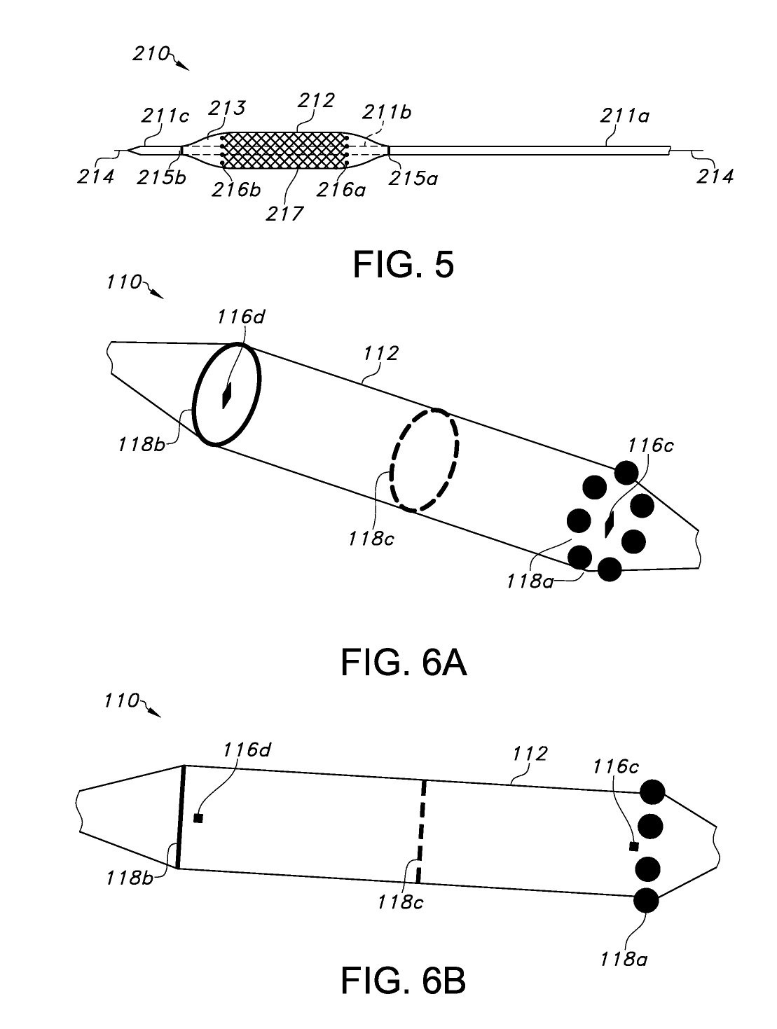

Now referring to FIGS. 4 & 5, another exemplary embodiment of the intravascular precision balloon catheter 210 of the present invention is shown, with (FIG. 5) and without (FIG. 4) stent 217. The components of precision balloon catheter 210 are essentially the same as that described above. For example, hollow shaft 211 is slidably engaged over a guidewire 214 to deliver an inflatable balloon member 212 with inner lumen 213 that is in fluid communication with at least one inner lumen of shaft 211. Shaft 211 has a proximal portion 211a (defined as the portion of shaft 211 that is proximal relative to the operator), a distal portion 211c (defined as the portion of shaft 211 that is distal relative to the operator), and a balloon portion 111b (defined as the portion of shaft 211 within or under the inflatable balloon member 212). Distal portion 211c is preferably tapered for entry into the patient's vessels. Shaft 211 may also comprise optional proximal shaft radiopaque marker 215a and a distal shaft radiopaque marker 215b.

Inflatable balloon member 212 is designed to expand at a lesion site to compress the plaque and thereby widening the patient's vessel. Inflatable balloon member 212 can be made of a non-compliant or semi-compliant polymeric material, as described above. The inflatable balloon member 212 at each end is sealingly connected to shaft 211 at the junctions of shaft portions 211a-211b and at 211b-211c so that the inflating fluid is maintained only within lumen 213. To ensure a proper angioplasty result, the inflating fluid is provided at several times normal blood pressure and only for a very brief period of time. Inflation causes inflatable balloon member 212 to reach a fixed or predetermined size at a given pressure that is proper for the artery and lesion being widened in the angioplasty procedure. Under-inflation by applying less than full pressure will cause inflatable balloon member 212 to not fully reach its predetermined size, which may be beneficial to a given application. The inflatable balloon member 212 and its seals must be capable of withstanding such extreme pressures, hold the predetermined size, and not burst.

As can be appreciated in FIGS. 4 & 5, a key difference between the precision balloon catheter 210 and balloon catheter 10 are the dual “rings” sets of proximal balloon spatial radiopaque markers 216a and distal balloon spatial radiopaque markers 216b placed circumferentially around balloon 212. When viewed orthogonally (as shown in FIGS. 4 & 5), proximal balloon spatial radiopaque markers 216a and distal balloon spatial radiopaque markers 216b each define a plane that is perpendicular to the length of shaft 211b associated with balloon 212. Proximal balloon spatial radiopaque markers 216a and distal balloon spatial radiopaque markers 216b may be positioned anywhere on balloon 212, but preferably proximal balloon spatial radiopaque markers 216a and distal balloon spatial radiopaque markers 216b are positioned at a site or sites of interest to the cardiac interventionist. For example, proximal balloon spatial radiopaque markers 216a and distal balloon spatial radiopaque markers 216b might be placed at the proximal and distal edges, respectively of the working area of balloon 212 (defined as the portion of the balloon that, when inflated, is designed to directly participate in the widening of the target vessel; see FIG. 4) or the proximal and/or distal end of stent 217 as placed on balloon 212 (see FIG. 5). Depending on the particular application site, stent 217 may or may not extend to the distal edge of the working area of balloon 212.

Now referring to FIGS. 6A & 6B, an embodiment of the precision balloon catheter 110 is shown under fluoroscopy (note: the edges of the balloon 112 are enhanced for clarity) with exemplary forms of sets of balloon spatial radiopaque markers 118a-c, that can be used as radiopaque markers 116 or 216. Balloon spatial radiopaque markers 118a-c can take any suitable shape or form. For example, and not intended to be limiting in any way, balloon spatial radiopaque markers 118a are a plurality of pellet-shaped metal objects fixedly attached to inflatable balloon member 112 by adhesive; balloon spatial radiopaque marker 118b is a solid ring-shaped non-metal object fixedly attached to inflatable balloon member 112 by adhesive; and balloon spatial radiopaque markers 118c are a plurality of curved linear-shaped metal objects fixedly attached to inflatable balloon member 112 by adhesive. The placement of balloon spatial radiopaque markers 118a at the proximal end, 118b at the distal end, and 118c in the working area of inflatable balloon member 112 is strictly coincidental, not intended to be limiting in any way, and only intended to show that many different shapes and forms of balloon spatial radiopaque markers 118a-c are acceptable on a balloon 112. Balloon spatial radiopaque markers 118a-c for use with precision balloon catheter 110 or 210 may be made of any radiopaque material, including metals (e.g., steel, gold, platinum, tantalum, alloys of the foregoing, and other suitable radiopaque metals or alloys) or flexible polymeric materials incorporating radiopaque filler materials (e.g., barium sulfate, bismuth compounds, and tungsten, or other suitable materials). Choice of radiopaque material will depend on the chosen method of placement, placement positioning, and the semi-compliant/non-compliant material of the balloon 112. Although shown as being fixedly attached by adhesive, radiopaque markers 118a-c can be fixedly attached to balloon 112 by any suitable means or method, including by embedding or incorporating the marker material into the balloon 112 material during manufacturing process. When adhesive is used, the adhesive is preferably a flexible adhesive such as a silicone-based adhesive, urethane-based adhesive, cyanoacrylate-based adhesive, or similar. For patient safety, it is preferred that any adhesively affixed radiopaque markers to balloon 112 or 212 are done so in the interior portion of the balloon. Because of the placement of balloon spatial radiopaque markers 118a-c on or in inflatable balloon member 112, it is critical that the marker material either is designed to expand with semi-compliant material during inflation of balloon 112 or is attached in such a way that does not impede inflation (for example, and in no way intended to be limiting, to attached when the balloon 112 material is at full inflation during manufacturing). For embodiments with a balloon 112 made of a non-compliant material, the ability of balloon spatial radiopaque markers 118a-c to expand during inflation is not as critical.

As can be appreciated in FIG. 6A, when the precision balloon catheter 110 is viewed under fluoroscopy at a non-orthogonal angle, the balloon spatial radiopaque markers 118a-c appear as “ring” structures around the circumference of inflatable balloon member 112. However, only when the precision balloon catheter 110 is viewed under fluoroscopy at an orthogonal angle (see FIG. 6B), the balloon spatial radiopaque markers 118a-c now “line up” and appear as “planar” structures perpendicular to the length of inflatable balloon member 112. Thus, the cardiac interventionist can easily determine whether the fluoroscopic image of the precision balloon catheter 110 is orthogonal or not. This has significant implications in the precise placement of a stent 117 at an aorto-ostial lesion. By ensuring that the precision balloon catheter 110 is being viewed at an orthogonal angle, the cardiac interventionist can have great confidence that the apparent plane of aorto-ostium junction is accurate in the two-dimensional fluoroscopic image without the added expense of additional balloon catheters, devices, guidewires, or IVUS imaging. Furthermore, by placing the proximal edge of stent 117 at the position of proximal balloon spatial radiopaque markers, e.g., 116 or 216, the cardiac interventionist can also confidently place stent 117 with unprecedented precision at the target ostial lesion site.

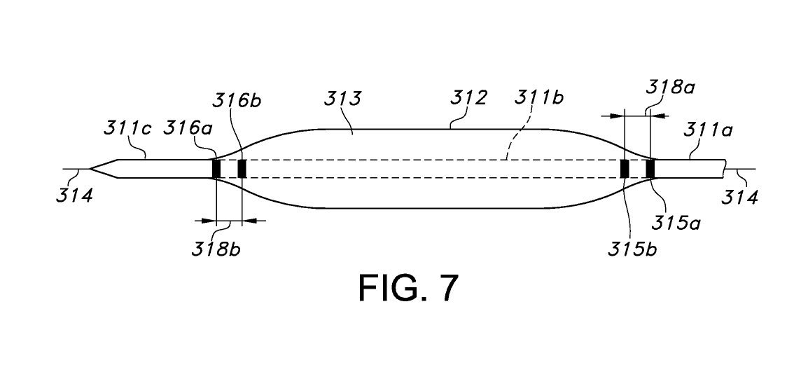

Now referring to FIG. 7, yet another exemplary embodiment of the intravascular precision balloon catheter 310 is shown. Although not shown with a stent, it should be understood that precision balloon catheter 310 is capable of delivering a stent as described above. The components of precision balloon catheter 310 are essentially the same as that described above. For example, hollow shaft 311 is slidably engaged over a guidewire 314 to deliver an inflatable balloon member 312 with inner lumen 313 that is in fluid communication with at least one inner lumen of shaft 311. Shaft 311 has a proximal portion 311a (defined as the portion of shaft 311 that is proximal relative to the operator), a distal portion 311c (defined as the portion of shaft 311 that is distal relative to the operator), and a balloon portion 311b (defined as the portion of shaft 311 within or under the inflatable balloon member 312). Distal portion 311c is preferably tapered for entry into the patient's vessels. Shaft 311 also comprises at least two proximal shaft spatial radiopaque markers 315a and 315b and, optionally, at least two distal shaft spatial radiopaque markers 316a and 316b.

Inflatable balloon member 312 is designed to expand at a lesion site to compress the plaque and thereby widening the patient's vessel. Inflatable balloon member 312 can be made of a non-compliant or semi-compliant polymeric material, as described above. The inflatable balloon member 312 at each end is sealingly connected to shaft 311 at the junctions of shaft portions 311a-311b and at 311b-311c so that the inflating fluid is maintained only within lumen 313. To ensure a proper angioplasty result, the inflating fluid is provided at several times normal blood pressure and only for a very brief period of time. Inflation causes inflatable balloon member 312 to reach a fixed or predetermined size at a given pressure that is proper for the artery and lesion being widened in the angioplasty procedure. Under-inflation by applying less than full pressure will cause inflatable balloon member 312 to not fully reach its predetermined size, which may be beneficial to a given application. The inflatable balloon member 312 and its seals must be capable of withstanding such extreme pressures, hold the predetermined size, and not burst.

As can be appreciated in FIG. 7, a key difference between the precision balloon catheter 310 and balloon catheter 10 are the sets of dual proximal shaft spatial radiopaque markers 315a and 315b and dual distal shaft spatial radiopaque markers 316a and 316b. The dual proximal shaft spatial radiopaque markers 315a/b and distal shaft spatial radiopaque markers 316a/b each have a known width on shaft 311 and are spaced apart at predetermined distances 318a & 318b, respectively, to each other. When viewed orthogonally (as shown in FIG. 7) under fluoroscopy, the width of and the distance between 318a proximal shaft spatial radiopaque markers 315a/b will appear to the viewer to be at a maximum. The same is true for optional distal shaft spatial radiopaque markers 316a/b if also employed. When viewed in a non-orthogonal angle, the widths and distances 318a & 318b will appear to become smaller. As such, the cardiac interventionist can have confidence that the fluoroscopy view is orthogonal or not. Also, proximal shaft spatial radiopaque markers 315a/b and distal shaft spatial radiopaque markers 316a/b may be positioned anywhere on shaft 311, but preferably at least one of proximal shaft spatial radiopaque markers 315a/b and optional distal shaft spatial radiopaque markers 316a/b are positioned at the junction of balloon 312 and shaft 311.

Although described in the context of precise positioning and delivery of balloon catheters and stents at aorto-ostial lesions that are inherently in arteries, it should be understood that embodiments of the present invention can also be used for precisely positioning a balloon catheter and/or stent at a venous site in need of widening or stent support.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. An inflatable balloon for angioplasty comprising an inflatable balloon member having a spatial radiopaque marker set circumferentially disposed around said inflatable balloon member.

2. The inflatable balloon for angioplasty of claim 1 further comprising an elongated shaft.

3. The inflatable balloon for angioplasty of claim 2, wherein said elongated shaft is disposed on a guidewire by slidable engagement.

4. The inflatable balloon for angioplasty of claim 3, wherein said guidewire comprises a hydrophilic material to facilitate movement within a vasculature system.

5. The inflatable balloon for angioplasty of claim 2, wherein said elongated shaft comprises at least one interior lumen in fluid communication with an inner chamber of said inflatable balloon member.

6. The inflatable balloon for angioplasty of claim 1, wherein said spatial radiopaque marker set is at least two spatial radiopaque marker sets.

7. The inflatable balloon for angioplasty of claim 2 further comprising at least one radiopaque marker disposed on said elongated shaft.

8. The inflatable balloon for angioplasty of claim 1 further comprising a stent.

9. The inflatable balloon for angioplasty of claim 8, wherein said stent comprises a drug releasing coating.

10. An intravascular balloon catheter comprising a hollow shaft member for extending a guidewire there through, said shaft member having at least one inner lumen for passing a fluid there through, an inflatable balloon member attached to said shaft member, an inner lumen of said inflatable balloon member in fluid communication with said at least one inner lumen of said shaft member for passing a fluid there through, and at least one spatial radiopaque marker set circumferentially disposed around said inflatable balloon member.

11. The intravascular balloon catheter of claim 10, wherein said shaft member is disposed on a guidewire by slidable engagement.

12. The intravascular balloon catheter of claim 11, wherein said guidewire comprises a hydrophilic material to facilitate movement within a vasculature system.

13. The intravascular balloon catheter of claim 10 further comprising at least one radiopaque marker disposed on said shaft member.

14. The intravascular balloon catheter of claim 10 further comprising a stent.

15. The intravascular balloon catheter of claim 14, wherein said stent comprises a drug releasing coating.

16. A method for making an intravascular balloon catheter with spatial radiopaque markers comprising: forming a hollow shaft member for extending a guidewire there through, said shaft member having at least one inner lumen for passing a fluid there through; forming an inflatable balloon member securely attached to said shaft member and leaving an inner lumen of said inflatable balloon member to be in fluid communication with said at least one inner lumen of said shaft member for passing a fluid there through; and incorporating a spatial radiopaque marker set circumferentially around said inflatable balloon member.

17. A method for making an intravascular balloon catheter with spatial radiopaque markers comprising: providing an intravascular balloon catheter with an inflatable balloon member; and incorporating a spatial radiopaque marker set circumferentially around said inflatable balloon member by flexible adhesive.

18. An intravascular balloon catheter comprising a hollow shaft member for extending a guidewire there through, said shaft member having at least one inner lumen for passing a fluid there through, an inflatable balloon member attached to said shaft member, an inner lumen of said inflatable balloon member in fluid communication with said at least one inner lumen of said shaft member for passing a fluid there through, and at least one pair of dual shaft spatial radiopaque markers disposed around said shaft member having a predetermined space between each marker of said at least one pair.

19. The intravascular balloon catheter of claim 18, wherein said at least one pair is at least two pairs.

20. The intravascular balloon catheter of claim 19, wherein said at least two pairs is a first pair disposed proximal to said inflatable balloon member and a second pair disposed distal to said inflatable balloon member.
